(19) United States
(12) Patent Application Publication
(10) Pub. No.: US 2025/0185964 A1
(43) Pub. Date: Jun. 12, 2025

(54) MULTIMODAL ELECTRIC AND MAGNETIC SENSOR

(71) Applicant: FieldLine Inc., Boulder, CO (US)

(72) Inventors: Kenneth J. Hughes, Lafayette, CO (US); Orang Alem, Erie, CO (US)

(21) Appl. No.: 18/535,539

(22) Filed: Dec. 11, 2023

(51) Int. Cl. A61B 5/245; A61B 5/06; A61B 5/263; A61B 5/291; G01R 33/26

(52) U.S. Cl. CPC A61B 5/245; A61B 5/062; A61B 5/263; A61B 5/291; G01R 33/26; A61B 2560/0443; A61B 2562/0217; A61B 2562/0223; A61B 2562/06

(57) ABSTRACT

Various embodiments comprise a multimodal electromagnetic sensing apparatus to detect neuronal activity in a target. In some examples, the multimodal electric and magnetic sensing apparatus comprises a magnetometer and an electrode. The magnetometer senses a target magnetic field generated by the neuronal activity in the target. The electrode senses a target electric field generated by the neuronal activity in the target. The electrode is coupled to the magnetometer and contacts the surface of the target.

### TECHNICAL FIELD

Various embodiments of the present technology relate to electromagnetic sensing, and more specifically, to multimodal sensors to detect electric and magnetic fields.

### BACKGROUND

Magnetometer systems detect and characterize magnetic fields generated by a magnetic field source. The magnetometer systems measure the field strength and/or direction of the magnetic fields to characterize the sensed fields. Magnetoencephalography (MEG) systems are a type of magnetometer system that measures magnetic fields generated by neuronal activity within the brain to map brain function. MEG systems image brain activity by detecting magnetic fields from neural currents using an array of magnetic sensors placed near the head of a patient. Exemplary magnetic sensors used in the MEG systems include Optically Pumped Magnetometers (OPMs), however other magnetometer types like Superconducting Quantum Interference Devices (SQUIDs) may be used. Electrode systems detect and characterize electric fields generated by an electric field source. The electrode systems measure voltage of the electric fields to characterize the sensed fields. Electroencephalography (EEG) systems are a type of electrode system that measures electric fields generated by neuronal activity within the brain to map brain function. EEG systems sense brain activity by detecting electric charges of neural currents using an array of electrodes adhered to the head of a patient.

It is often desirable to perform both MEG and EEG on a patient. In conventional systems for simultaneous MEG and EEG, an EEG electrode array is adhered to the scalp of the patient. The patient then places their head within a SQUID system which measures magnetic fields generated by the brain of the patient. The EEG electrodes and the SQUID system report the sensed electric and magnetic fields to a controller. SQUID systems are large devices and require the patient to remain stationary while measurements take place. An alternative to SQUID systems is conformal MEG. In conformal MEG, headgear that mounts magnetometers is worn by the patient. Conformal MEG systems are smaller and do not restrict the mobility of the patient when compared to SQUID based MEG. However, performing simultaneous MEG and EEG using a conformal MEG system is cumbersome. The EEG electrode array may interfere with the MEG headgear. For example, the electrode array may inhibit magnetometer contact with the head of the patient thereby reducing the measurement fidelity of the magnetometers. Having the patient wear both the electrode array and the MEG headgear may be uncomfortable for the patient.

Unfortunately, conventional simultaneous EEG/MEG systems are cumbersome. Moreover, conventional simultaneous EEG/MEG do not effectively contact both EEG electrodes and MEG magnetometers with the patient.

### Overview

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to magnetic and electric field sensing. Some embodiments comprise a multimodal electric and magnetic sensing apparatus to detect neuronal activity in a target. The apparatus comprises a magnetometer and an electrode. The magnetometer senses a target magnetic field generated by the neuronal activity in the target. The electrode senses a target electric field generated by the neuronal activity in the target. The electrode is coupled to the magnetometer and contacts the surface of the target.

Some embodiments comprise multimodal sensing system to detect neuronal activity in a target. The system comprises a sensor mount, a multimodal sensor array comprising magnetometers and electrodes, and a controller. The sensor mount mounts the multimodal sensor array. The magnetometers sense a target magnetic field generated by the neuronal activity in the target. The electrodes contact the surface of the target and sense a target electric field generated by the neuronal activity in the target. Each of the electrodes is coupled to a corresponding one of the magnetometers. The controller is communicatively coupled to the magnetometers and the electrodes. The controller processes signaling received from the magnetometers and the electrodes to characterize the target magnetic and electric fields.

Some embodiments comprise a method of operating a multimodal sensing system to detect neuronal activity in a target. The method comprises a sensor mount mounting a multimodal sensor array comprising magnetometers and electrodes. The method further comprises the magnetometers sensing a target magnetic field generated by the neuronal activity in the target. The method further comprises the electrodes contacting the surface of the target and sensing a target electric field generated by the neuronal activity in the target. Each of the electrodes is coupled to a corresponding one of the magnetometers. The method further comprises a controller processing signaling received from the magnetometers and the electrodes to characterize the target magnetic field and the target electric field.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary multimodal sensor.

FIG. 2 illustrates further illustrates the multimodal sensor.

FIG. 3 illustrates an exemplary multimodal sensing system.

FIG. 4 illustrates an exemplary operation of the multimodal sensing system.

FIG. 5 illustrates an exemplary multimodal sensor.

FIG. 6 further illustrates the multimodal sensor.

FIG. 7 further illustrates the multimodal sensor.

FIG. 8 illustrates exemplary electrode arrangements for a multimodal sensor.

FIG. 9 illustrates an exemplary multimodal Electroencephalography/Magnetoencephalography (EEG/MEG) system.

FIG. 10 illustrates an exemplary operation of the EEG/MEG system.

FIG. 11 further illustrates the multimodal EEG/MEG system.

FIG. 12 illustrates an exemplary operation of the EEG/MEG system.

FIG. 13 illustrates an exemplary computing apparatus.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

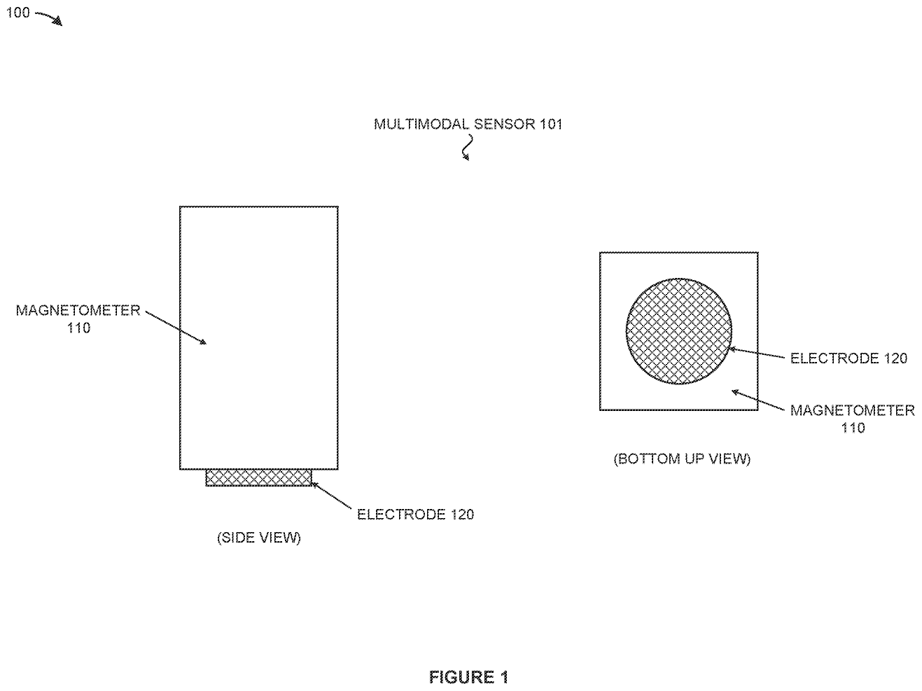

FIG. 1 comprises view 100. View 100 illustrates multimodal sensor 101 in a side and bottom-up perspective. Multimodal sensor 101 performs operations like magnetic field sensing and electric field sensing. Multimodal sensor 101 comprises magnetometer 110 and electrode 120. In other examples, multimodal sensor 101 may include fewer or additional components than those illustrated in FIG. 1. Likewise, the illustrated components of multimodal sensor 101 may include fewer or additional components, assets, or connections than shown.

Various examples of sensor operation and configuration are described herein. In some examples, electrode 120 is coupled to the surface of magnetometer 110 to form multimodal sensor 101. For example, magnetometer 110 may comprise an Optically Pumped Magnetometer (OPM) and electrode 120 may comprise an Electroencephalography (EEG) electrode. Electrode 120 comprises a circular and electrically-conductive disk. When in contact with a target (e.g., a human scalp), electrode 120 detects electric fields in the target that it is contacting. Magnetometer 110 comprises circuitry sensitive to the magnetic component of an electromagnetic field. When placed proximate to a target (e.g., the human brain), magnetometer 120 detects magnetic fields in the target.

Electrode 120 is coupled to magnetometer 110 using an adhesive, male/female socket connection, a threaded connection, hook-an-loop fasteners, and the like. This coupling allows multimodal sensor 101 to be used in simultaneous (or sequential) magnetic and electric field detection. In some examples, sensor 101 includes a magnetometer cap to couple electrode 120 to magnetometer 110. For example, the magnetometer cap may fit over the outer surface of magnetometer 110 and electrode 120 may be embedded into the surface of the cap. In some examples, electrode 120 is instead mounted internally in magnetometer 110 where a sensing portion of electrode 120 protrudes (or is otherwise exposed) from magnetometer 110. Magnetometer 110 may comprise an atomic magnetometer, an OPM, a gradiometer, a nitrogen vacancy center, a high-temperature Superconducting Quantum Interference Devices (SQUID), or some other type of magnetic sensing device. Although electrode 120 is illustrated as a single disk, the geometry and number of contact points of electrode 120 may differ in other examples. For example, electrode 120 may comprise four square shaped electrodes attached to the surface of magnetometer 110. Magnetometer 110 and electrode 120 typically comprise external communication circuitry like cabling and/or wireless transceivers to receive sensing instructions and transfer signaling that characterizes the sensed electric and magnetic fields to external systems.

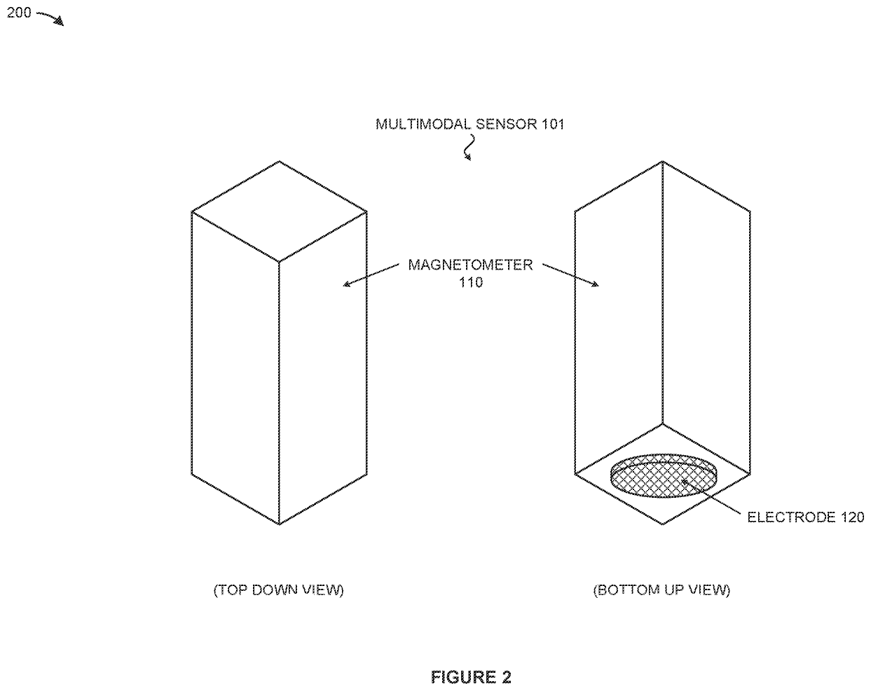

FIG. 2 comprises view 200. View 200 illustrates multimodal sensor 101 in a top-down and bottom-up perspective. As illustrated in FIG. 2, electrode 120 is coupled to the bottom surface of magnetometer 110. Magnetometers 110 may comprise a sensing surface that is pointed at the target. For example, the magnetic sensing instruments (e.g., vapor cell) in a magnetometer may be internally mounted to the bottom surface of the magnetometer. As such, pointing the bottom surface of the magnetometer at the target decreases the distance between the magnetic field source of the target and the magnetic sensing instruments. When magnetometer 110 comprises a sensing surface, electrode 120 may be mounted to the sensing surface so that when electrode 120 is contacted with a target, the proximity between the magnetic sensing instruments in magnetometer 110 and the magnetic field source of the target are reduced. When magnetometer 110 does not comprise a sensing surface, electrode 120 may be mounted to any external surface of magnetometer 110.

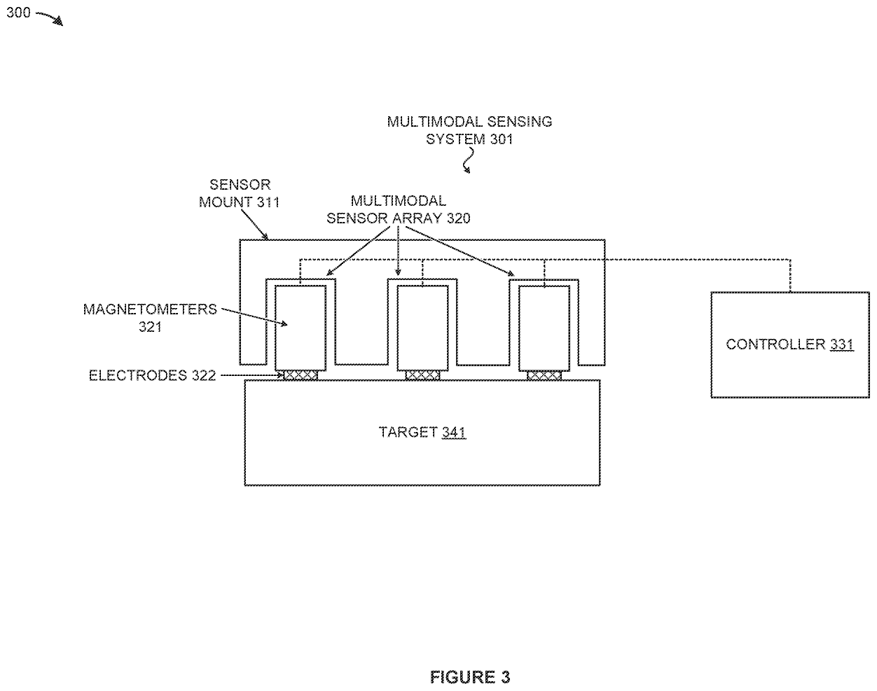

FIG. 3 comprises view 300. View 300 illustrates multimodal sensing system 301. Multimodal sensing system 301 performs operations like detecting magnetic and electric fields and relating the detected magnetic and electric fields to neuronal activity for use in medical applications. Exemplary medical applications include identifying brain activity and diagnosing medical conditions like stroke, epilepsy, neuronal injuries, neuronal disorders, and/or other types of medical conditions relating to brain/neuron activity. Multimodal sensing system 301 comprises sensor mount 311, multimodal sensing array 320, controller 331, and target 341. Multimodal sensor array 320 comprises magnetometers 321 and electrodes 322. In other examples, multimodal sensing system 301 may include fewer or additional components than those illustrated in FIG. 3. Likewise, the illustrated components of multimodal sensing system 301 may include fewer or additional components, assets, or connections than shown.

In some examples, sensor mount 311 comprises an apparatus to mount multimodal sensor array 320. Mount 311 may comprise a rigid helmet, a flexible hat, a blanket, a sleeve, a vest, and the like. Typically, mount 311 is wearable by target 341. For example, if target 341 comprises an adult human, mount 311 may be shaped to fit over part of the human body (e.g., the head). Mount 311 may be contrasted from plastic, carbon fiber, polymer, rubber, fabric, canvas, or other materials that provide structural support to mount 311 and that do not interfere in the sensing operations of array 320. As illustrated in FIG. 3, mount 311 comprises slots for sensor array 320. The multimodal sensors of array 320 fit into the slots to connect to mount 311. The multimodal sensors of array 320 may connect to mount 311 using male/female sockets, ratchet mechanisms, set screws, springs, pistons, pneumatics, and the like. The connectors between array 320 and mount 311 may be adjustable or static. Mount 311 may comprise embedded circuitry to communicatively couple array 320 and controller 341. Alternatively, mount 311 may comprise conduit (or another type of passage) that allows wiring to communicatively couple array 320 and controller 341.

Multimodal sensor array 320 comprises magnetometers 321 and electrodes 322 to measure magnetic and electric fields generated by target 341. For example, when target 341 comprises a human, multimodal sensor array 320 may sense electric and magnetic fields generated by the brain, heart, muscles, and the like in target 341. Multimodal sensor array 320 may perform Electroencephalography (EEG), Electrocardiogramactromyography (EMG), Magnetoencephalography (MEG), Magnetocardiography (MCG), Magnetogastrography (MGG), Magnetomyography (MMG), and/or other types of anatomical electric and magnetic field sensing operations. For example, sensor array 320 may perform simultaneous EEG/MEG on target 341. It should be appreciated that target 341 may comprise any electric and magnetic field source (e.g., the human brain), including non-biological field sources.

Magnetometers 321 and electrodes 322 comprise examples of multimodal sensor 101 illustrated in FIG. 1, however sensor 101 may differ. Electrodes 322 are coupled to the surface of magnetometers 321 and contact the surface of target 341. Magnetometers 321 may comprise atomic magnetometers, OPMs, gradiometers, nitrogen vacancy centers, a high-temperature SQUIDs, and/or other types of magnetic sensing devices. Magnetometer 321 may comprise subcomponents like atomic vapor cells, lasers, heaters, bias coils, photodetectors, processing circuitry, and communication circuitry. Electrodes 322 may comprise gold-plated electrodes, sponge electrodes, dry electrodes, and/or other types of electrodes. Electrodes 322 are typically constructed from non-magnetic material to not interfere with the magnetic sensing operations of magnetometers 321. Sponge electrodes are typically soaked in an electrolyte solution (e.g., saline) to provide the electric sensing capability.

Controller 341 comprises one or more computing devices to control the operation of sensor array 320 to sense electric and magnetic fields generated by target 341 and process signaling received from array 320. Controller 341 is communicatively coupled to magnetometers 321 and electrodes 322. The communication links between controller 341 and array 320 may comprise metallic links, glass fibers, radio channels, or some other communication media. The links may use inter-processor communication, bus interfaces, Ethernet, WiFi, virtual switching, and/or some other communication protocol. For example, the communication links may be supported by sheathed metallic wires to exchange signaling between controller 341 and array 320. Alternatively, the communication links may be supported by a wireless transceiver (e.g., antennas) to exchange signaling between controller 341 and array 320 over a wireless networking protocol like Bluetooth. Controller 341 may supply electric current over a wired connection to power array 320. Alternatively, array 320 may be battery powered.

The one or more computing devices of controller 341 comprise processors, memories, and transceivers that are connected over bus circuitry. The processors may comprise Central Processing Units (CPUs), Graphical Processing Units (GPUs), Digital Signal Processors (DSPs), Application Specific Integrated Circuits (ASICs), Field Programmable Gate Arrays (FPGAs), and the like. The memories may comprise Random Access Memory (RAM), flash circuitry, Solid States Drives (SSDs), Hard Disk Drives (HDDs), and the like. The memory stores software like operating systems, MEG applications, localization applications, sensor data, and the like. The processors retrieve and execute the software from the memory to drive the operation of controller 341.

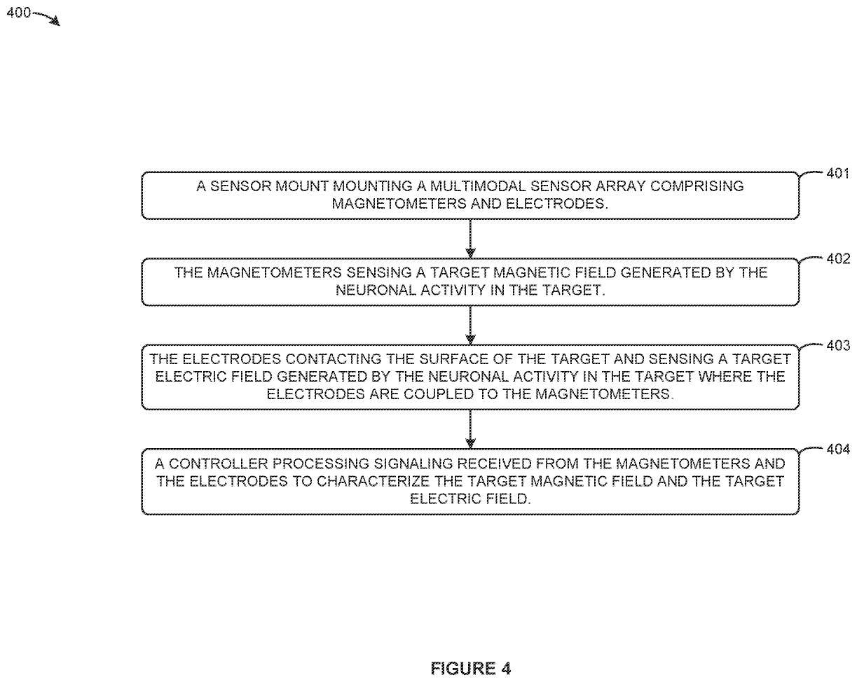

In some examples, multimodal sensing system 301 implements process 400 illustrated in FIG. 4. It should be appreciated that the structure and operation of system 301 may differ in other examples.

FIG. 4 illustrates process 400. Process 400 comprises an exemplary multimodal sensing process. In other examples, process 400 may differ. The operations of process 400 comprise a sensor mount mounting a multimodal sensor array comprising magnetometers and electrodes (step 401). The operations further comprise the magnetometers sensing a target magnetic field generated by the neuronal activity in the target (step 402). The operations further comprise the electrodes contacting the surfaces of the target and sensing a target electric field generated by the neuronal activity in the target where the electrodes are coupled to the magnetometers (step 403). The operations further comprise a controller processing signaling received from the magnetometers and the electrodes to characterize the target magnetic field and the target electric field (step 404).

Referring back to FIG. 3, multimodal sensing system 301 includes a brief example of process 400 as employed by the various components of system 301. In some examples, target 341 comprises a human and multimodal sensing system 301 performs EEG and MEG to sense electric and magnetic fields generated by neuronal activity in the brain of target 341. Sensor mount 311 mounts array 320 in its sensor slots (step 401). Mount 311 is placed onto the head of target 341 and is adjusted to contact electrodes 322 with the surface of target 341. For example, mount 311 may comprise adjustable ratchet mechanisms that may propel the multimodal sensors of array 320 through their respective slots until electrodes 322 contact target 341. Contacting electrodes 322 to target 341 also places magnetometers 321 at spatial locations proximate to target 341.

Once array 320 is in place, controller 341 generates and transfers instructions to magnetometers 321 to measure the magnetic field generated by neuronal activity in the brain of target 341. Magnetometers 321 measure the strength of target 341's magnetic field and transfer signaling that indicates the measured field strengths to controller 331 (step 402). Controller 331 records the measured field strengths for target 341. When electrodes 322 contact the surface of target 341, the electric field generated by neuronal activity in the brain of target 341 interacts with electrodes 322. The electric field applies a voltage to electrodes 322. Electrodes 322 indicate the sensed voltages to controller 331 (step 403). Controller 331 measures voltage differences between any two of electrodes 322 and record the voltage differences for the EEG of target 341. The EEG may be performed before, during, or after magnetometers 321 sense the magnetic field. Controller 331 processes the MEG and EEG data to characterize target 341's electric and magnetic fields (step 404). For example, controller 331 may construct an MEG image that depicts neuronal activity based on the measured field strengths and may plot the EEG data over time to construct an electroencephalogram for target 341.

Advantageously, multimodal sensing system 301 efficiently measures electric and magnetic fields generated by a target. Moreover, multimodal sensing system 301 effectively contacts both electrodes and magnetometers with the target by adhering the electrodes to the magnetometers. By combining the magnetometers and electrodes, a separate electrode array does not need to be adhered to the target. This increases the comfort of the target and allows for efficient conformal MEG/EEG.

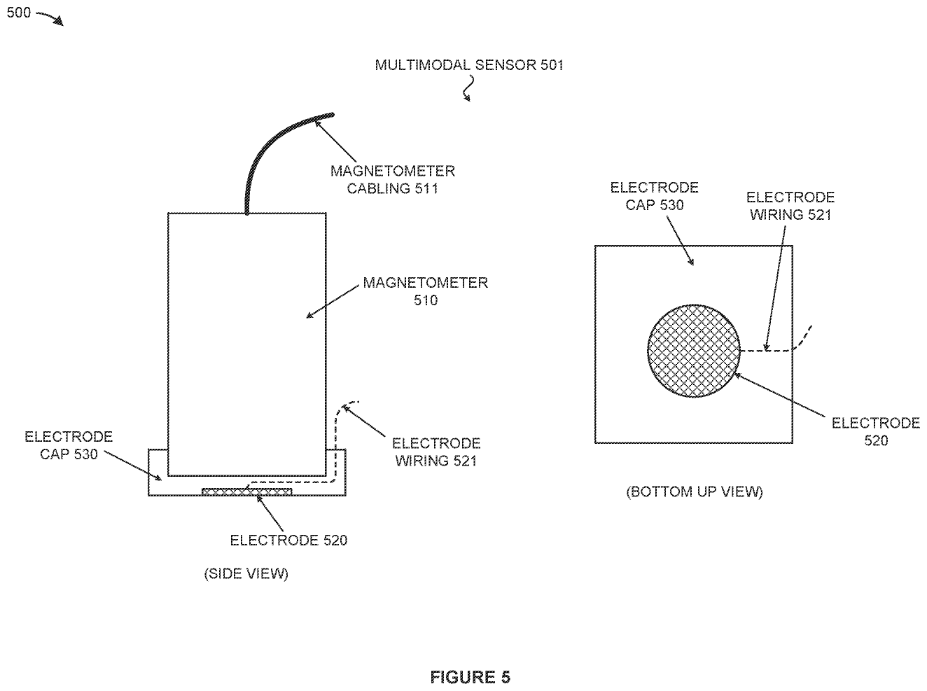

FIG. 5 comprises view 500. View 500 illustrates multimodal sensor 501 in a side and bottom-up perspective. Multimodal sensor 501 comprises an example of multimodal sensor 101 illustrated in FIGS. 1 and 2 and the sensors that compose multimodal sensor array 320 illustrated in FIG. 3, however sensor 101 and array 320 may differ. Sensor 501 comprises magnetometer 510, magnetometer cabling 511, electrode 520, electrode wiring 521, and electrode cap 530. In other examples, multimodal sensor 501 may include fewer or additional components than those illustrated in FIG. 5. Likewise, the illustrated components of multimodal sensor 501 may include fewer or additional components, assets, or connections than shown.

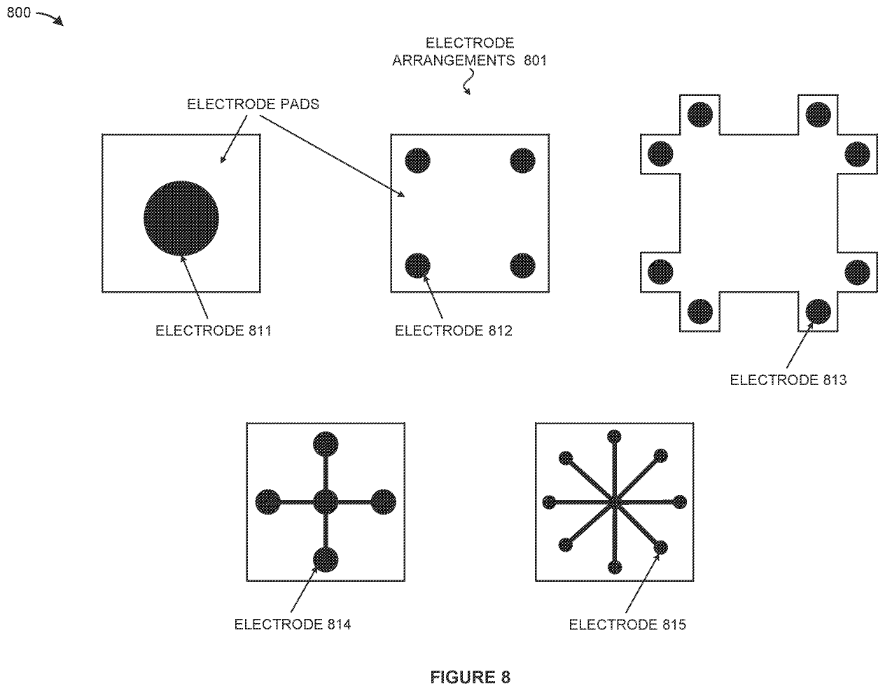

In some examples, electrode 520 comprises a conductor that senses electric fields when contacted to the surface of field source (e.g., the human body). Electrode 520 may comprise a gold-plated electrode, copper-plated electrode, a sponge electrode, a dry electrode, or some other type of non-magnetic conductor. Electrode 520 is embedded into the surface of electrode cap 530. Although electrode 520 is illustrated as a circular electrode, the geometry of electrode 520 may differ in other examples. For example, electrode 520 may comprise a star shaped surface geometry similar to electrode 815 illustrated in FIG. 8. Magnetometer 510 comprises a magnetic sensing device to sense magnetic fields when placed in proximity to a magnetic field source. Magnetometer 510 may comprise an atomic magnetometer, OPM, gradiometer, nitrogen vacancy center, a high-temperature SQUID, and/or another type of magnetic sensing device. Magnetometer cabling 511 and wiring 521 couple magnetometer 510 and electrode 520 to external systems (e.g., an EEG/MEG controller). Cabling 511 and wiring 521 comprise sheathed metallic wires. Cabling 511 and/or wiring 521 may be detachably coupled to magnetometer 510 and electrode 520 respectively.

Electrode cap 530 is representative of a mounting element to couple electrode 520 to magnetometer 510. Cap 530 may be constructed of a material like silicone, rubber, plastic, carbon fiber, and/or some other material that does not inhibit the sensing operations of magnetometer 510 and electrode 520. For example, magnetic materials like iron may not be a suitable material to construct cap 530 as this could interfere with the sensing operations of magnetometer 510. Wiring 521 is partially embedded within cap 530. The geometry of cap 530 aligns with the geometry of magnetometer 510. Cap 530 fits over the outer surface of magnetometer 510 to couple electrode 520 to magnetometer 510. Cap 530 may attach to magnetometer 510 using an adhesive, a male/female socket connection, a threaded connection, hook-an-loop fasteners, and the like. For example, cap 530 may fit snuggly over magnetometer 530 and the resulting surface friction between cap 530 and magnetometer 510 inhibits cap 530 from detaching from magnetometer 510. In this example, magnetometer 510 is rectangular in shape. Accordingly, electrode cap 530 is similarly shaped to fit over magnetometer 510. It should be apperceived that in other examples, the shape of magnetometer 510 and cap 530 may differ. For example, magnetometer 510 may comprise a cylindrical shape. Consequently, cap 530 may be circular in shape and the inner diameter of cap 530 may match the outer diameter of magnetometer 510.

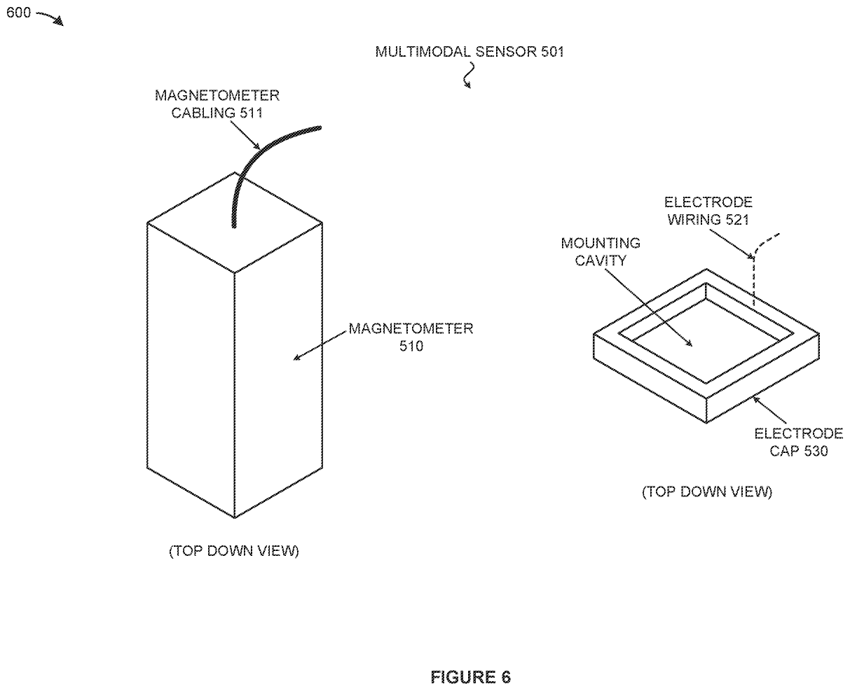

FIG. 6 comprises view 600. View 600 illustrates multimodal sensor 501 from a top-down perspective. Magnetometer 510 and cap 530 are illustrated in their detached state. The dimensions of the mounting cavity of cap 530 align with the exterior dimensions of magnetometer 510. Magnetometer 510 may be inserted into the mounting cavity to couple electrode cap 530 with magnetometer 510. For example, an epoxy may be applied in the mounting cavity of cap 530 and magnetometer 510 may be inserted into the mounting cavity and coupled to cap 530 when the epoxy cures.

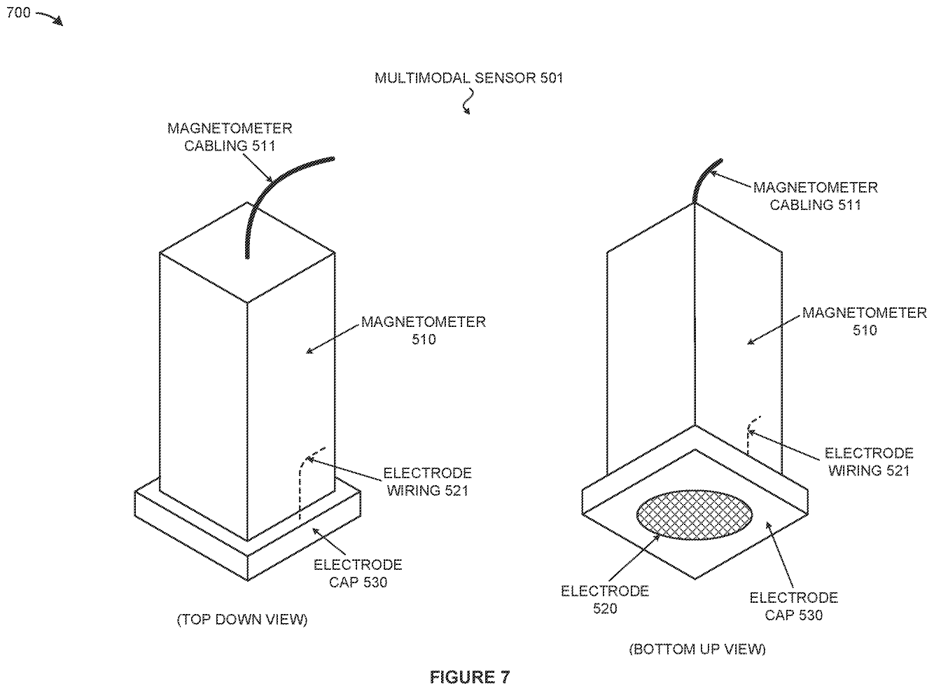

FIG. 7 comprises view 700. View 700 illustrates multimodal sensor 501 from a top-down perspective and bottom-up perspective. Magnetometer 510 and cap 530 are illustrated in their attached state. As illustrated in FIG. 7, the sensing surface of electrode 520 is exposed by cap 530. This exposure allows electrode 520 to contact a target surface and sense electric fields generated by a target.

FIG. 8 comprises view 800. View 800 illustrates electrode arrangements 801. Electrode arrangements 801 comprises electrodes 811-815 and are representative of different electrode geometries according to various embodiments of the present technology. Electrodes 811-815 comprise an example of electrodes 120, 322, and 520 illustrated in FIGS. 1-3 and 5, however electrodes 120, 322, and 520 may differ. Each of electrodes 811-815 are embedded into an electrode cap that fits over a corresponding magnetometer. The electrode caps may be attached to the corresponding magnetometers to couple electrode 811-805 to the magnetometers to form multimodal sensing devices. Electrode 811 comprises a centrally positioned circular electrode. Electrode 812 comprises four circular electrodes positioned on the corners of the electrode cap. Electrode 813 comprise eight circular electrodes in an offset orientation. The offset orientation allows the thickness of the electrode cap to be reduced. The reduced thickness decreases the distance between the magnetometer and the magnetic field source thereby increasing the measurement fidelity of the magnetometer. Electrode 814 comprises four circular electrodes positioned in a cross along the central axes of the electrode pad. Electrode 815 comprises nine electrodes arranged in a circular or star formation. Electrodes 814 and 815 comprise crosslinks to link each of the individual pads.

In should be appreciated that circular electrodes (e.g., electrode 811) may allow eddy currents to form. An eddy current is a loop of electric current induced within a conductor by changing electric field. The circular shape of some electrodes allows eddy currents to travel around the electrode. Eddy currents create Johnson noise which degrades the signal produced by electrodes (e.g., degrade EEG measurements). The cross and star shapes of electrodes 814 and 815 inhibit the formation of eddy currents by inhibiting the circular flow of electrons. Inhibiting eddy currents reduces or eliminates Johnson noise which increases the quality of the signal produced by the electrodes.

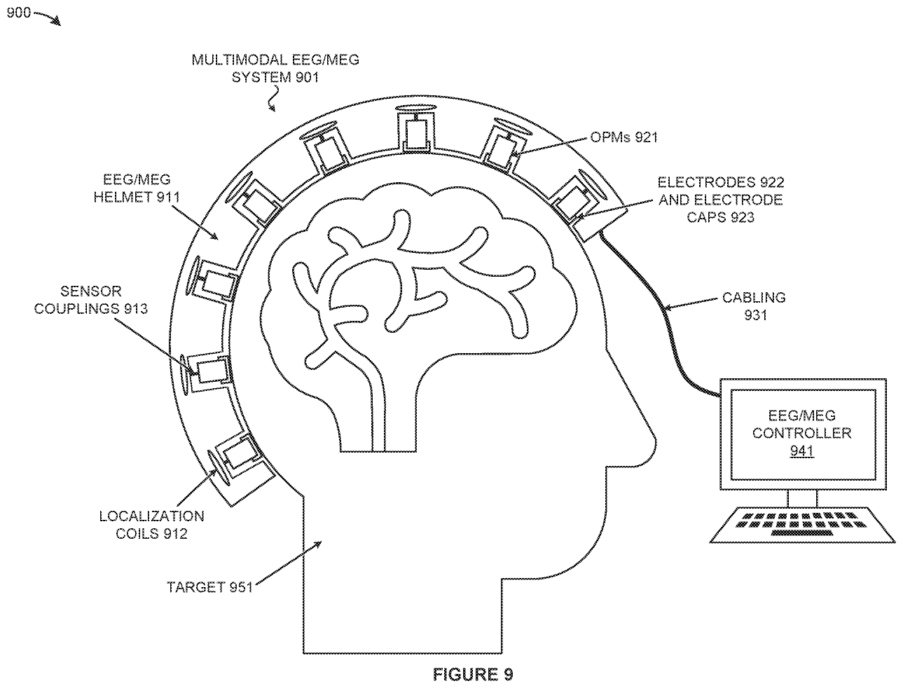

FIG. 9 comprises view 900. View 900 illustrates multimodal EEG/MEG system 901. EEG/MEG system 901 comprises an example of multimodal sensing system 301, however system 301 may differ. EEG/MEG system 901 is an example of a conformal EEG/MEG system and performs operations like detecting electric and magnetic fields and relating the detected fields to neuronal activity for use in medical applications. Exemplary medical applications include identifying brain activity and diagnosing conditions like stroke, epilepsy, brain injuries, brain disorders, and/or other types of medical conditions relating to brain/neuron activity. MEG/EEG system 901 comprises EEG/MEG helmet 911, OPMs 921, electrodes 922, electrode caps 923, cabling 931, controller 941, and target 951. Helmet 911 comprises localization coils 912 and sensor couplings 913. In other examples, EEG/MEG system 901 may include fewer or additional components than those illustrated in FIG. 9. Likewise, the illustrated components of system 901 may include fewer or additional components, assets, or connections than shown.

In some examples, helmet 911 comprises a conformal EEG/MEG apparatus. Helmet 911 is shaped to conform to the geometry of a human head. Helmet 911 is wearable by target 951 and positions OPMs 921 and electrodes 922 in locations proximate to target 951. For example, helmet 911 may securely adhere the multimodal sensors to the scalp of target 951 using mechanical constraints. Helmet 911 may be contrasted from rigid plastic, carbon fiber, polymer, or other types of materials that provide structural support to helmet 911 and that do not interfere in the magnetic and electric sensing operations of the multimodal sensors. Helmet 911 comprises slots that form channels to control the position and orientation of OPMs 921 and electrodes 922. For example, the slots may be shaped to constrain the three orientational degrees of freedom for each of the multimodal sensors and two of the three locational degrees of freedom for each of the multimodal sensors allowing for each of the multimodal sensors to move along a single axis of motion. Helmet 911 may comprise support elements like padding, straps, cushions, and/or some other type of the support system to support and position the head of target 951 within helmet 911.

Sensor couplings 913 attach OPMs 921 and electrodes 922 to helmet 911. The couplings control one or more degrees of freedom in the position and orientation of OPMs 921 and electrodes 922. Sensor couplings 913 may comprise ratchet mechanisms, set screws, springs, pistons, pneumatics, and the like. As stated above, the slots are shaped to constrain the three orientational degrees of freedom and two of the three locational degrees of freedom for each of the multimodal sensors. Sensor couplings 913 control the last locational degree of freedom for each of the multimodal sensors. When helmet 911 is worn by target 911, the sensor couplings 913 may propel the multimodal sensors through their respective slots to contact electrodes 922 with target 951. For example, set screws in couplings 913 may be tightened to move the multimodal sensors. Once electrodes 922 contact target 951, sensor couplings 913 may lock to secure the multimodal sensors. Once locked, all six of the orientational and locational degrees of freedom for OPMs 921 and electrodes 922 are fixed.

Helmet 911 mounts localization coils 912. Coils 912 comprise loops of metallic wiring that generate an electromagnetic field in response to receiving electric current. For example, coils 912 may comprise copper or aluminum wiring. Coils 912 may comprise single or multiple loops of any shape and size. Coils 912 may comprise sets of separated coils with differing loops of varying shapes, sizes, and orientations. Coils 912 are embedded into the surface of helmet 911. Coils 912 are stationary with respect to each other. Individual ones of coils 912 correspond to individual ones of OPMs 921 on a one-to-one basis. When powered, coils 912 generate magnetic waves that form coil magnetic fields. OPMs 921 may measure the coil magnetic fields and report the field strength to controller 941. Controller 941 may determine the location of OPMs 921 based on the reported field strengths, the orientational and locational constraints, and the locations of coils 912. Controller 941 may also determine the locations of electrodes 922 based on the locations of OPMs 921.

OPMs 921 and electrodes 922 comprise multimodal sensors to sense electric and magnetic fields generated by brain activity of target 951 for simultaneous EEG and MEG. OPMs 921 also sense magnetic fields generated by coils 912 during localization. OPMs 921 and electrodes 922 generate signals that characterize the strength of the sense electric and magnetic fields. The neuronal activity in the brain of target 951 comprises intercellular electromagnetic signals. OPMs 921 sense the magnetic component of the electromagnetic signals to detect neuronal activity. Likewise, electrodes 922 sense the electric component of the electromagnetic signals to detect neuronal activity. OPMs 921 and electrodes 922 form a sensor array that is contoured to the head of target 951 by helmet 911. In this example, electrodes 922 comprise gold-plated EEG electrodes and embedded into the surface of electrode caps 923. In other examples, the electrode type may differ. Electrode caps 923 rubber pieces that fit over OPMs 921 to couple electrodes 922 to OPMs 921. For example, OPMs 921 and electrodes 922 may comprise a similar structure multimodal sensor 501 illustrated in FIG. 5.

OPMs 921 and electrodes 922 are coupled to controller 941 over cabling 931. Cabling 931 comprises sheathed metallic wires. For example, OPMs 921 and electrodes 922 may transfer signaling that characterizes the sensed electric and magnetic fields to controller 941 over cabling 931. In some examples, cabling 931 may be replaced with, or used in addition with, a wireless transceiver system (e.g., antennas) to transfer communications between controller 941 and the multimodal sensors over a wireless networking protocol like Bluetooth.

Controller 941 is representative of one or more computing devices configured to drive the operation of OPMs 921, electrodes 922, and coils 912, localize OPMs 921 and electrodes 922, and generate electroencephalograms and MEG images that depict the neuronal activity in target 951. The one or more computing devices comprise processors, memories, and transceivers that are connected over bus circuitry. The processors may comprise CPUs, GPUs, DSPs, ASICS, FPGAs, and the like. The memories may comprise RAM, HDD, SSD, NVMe SSD, and the like. The memory stores software like operating systems, control application, localization applications, MEG applications, EEG applications, sensor data, and the like. The processors retrieve and execute the software from the memory to drive the operation of controller 941.

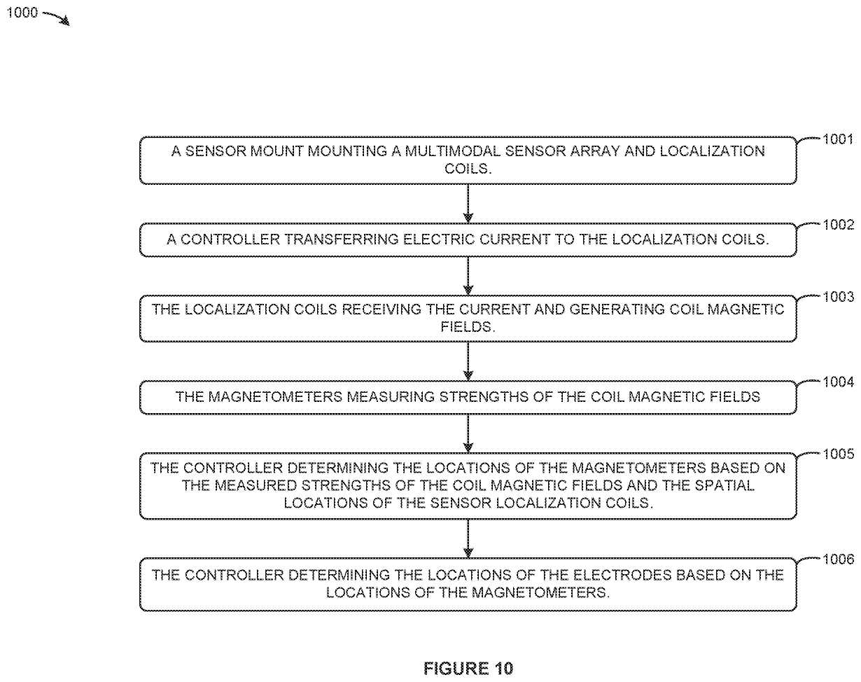

In some examples, EEG/MEG system 901 implements process 1000 illustrated in FIG. 10. It should be appreciated that the structure and operation of EEG/MEG system 901 may differ in other examples.

FIG. 10 illustrates process 1000. Process 1000 comprises an exemplary multimodal sensor localization process. In other examples, process 1000 may differ. The operations of process 1000 comprise a sensor mount mounting a multimodal sensor array and localization coils (step 1001). The operations further comprise a controller transferring electric current to the localization coils (step 1002). The operations further comprise the localization coils receiving the current and generating coil magnetic fields (step 1003). The operations further comprise the magnetometers measuring strengths of the coil magnetic fields (step 1004). The operations further comprise the controller determining the locations of the magnetometers based on the measured strengths of the coil magnetic fields and the spatial locations of the sensor localization coils (step 1005). The operations further comprise the controller determining the locations of the electrodes based on the locations of the magnetometers (step 1006).

Referring back to FIG. 9, EEG/MEG system 901 includes a brief example of process 1000 as by the various components of EEG/MEG system 901. In some examples, helmet 911 is placed on the head of target 951 and sensor couplings 913 are adjusted to drive OPMs 921 and electrodes 922 through their respective slots until electrodes 922 contact the scalp of target 951 (step 1001). Once OPMs 921 and electrodes 922 are conformed to the shape of target 951, controller 941 supplies electric current to one of coils 912 (step 1002). The one of coils 912 responsively generates magnetic waves that form a coil magnetic field (step 1003). The coil magnetic field may comprise any frequency or phase and may comprise a homogenous magnetic field or a gradient magnetic field. A homogenous magnetic field is constant in both field magnitude and direction over a region of interest while a gradient magnetic field varies in either or both magnitude and direction over a region of interest. Controller 941 identifies the one of OPMs 921 that corresponds to the activated one of coils 912 and transfers instructions to the OPM to measure field strength of the coil magnetic field. The OPM receives the instructions and responsively measures the coil magnetic field and reports sensor data characterizing the coil magnetic field to controller 941 (step 1004). The sensor data may comprise field strength, measured field gradient, field orientation and direction, and/or other attributes of the coil magnetic field.

Controller 941 correlates the reported magnetic field characteristics and the spatial location of the activated coil to the location and/or orientation of the corresponding OPM (step 1005). For example, the spatial location of the coil may comprise a reference point known by controller 941. Controller 941 may correlate the measured field strength to a distance between the coil and the corresponding OPM. Controller 941 may then calculate the spatial location of the OPM based on the spatial location of the coil, the correlated distance, and the positional/orientational constraints applied to the OPM by its slot and sensor coupling. Once the location of the OPM is determined, controller 941 determines the spatial location of the one of electrodes 922 coupled to the OPM based on the location of the OPM (step 1006). Since the OPM and its corresponding electrode are coupled, controller 941 may simply designate the two as being co-located. Alternatively, controller 941 may more precisely determine the spatial location of the electrode. For example, controller 941 may determine the location of the electrode based on the location of its OPM, the location of the vapor cell within the OPM, and the distance between the electrode and the vapor cell. Controller 941 may repeat the above process to locate each of OPMs 921 and electrodes 922. The multimodal sensor locations may be used for source localization to generate electroencephalograms and MEG images to illustrate the neuronal activity in the brain of target 951.

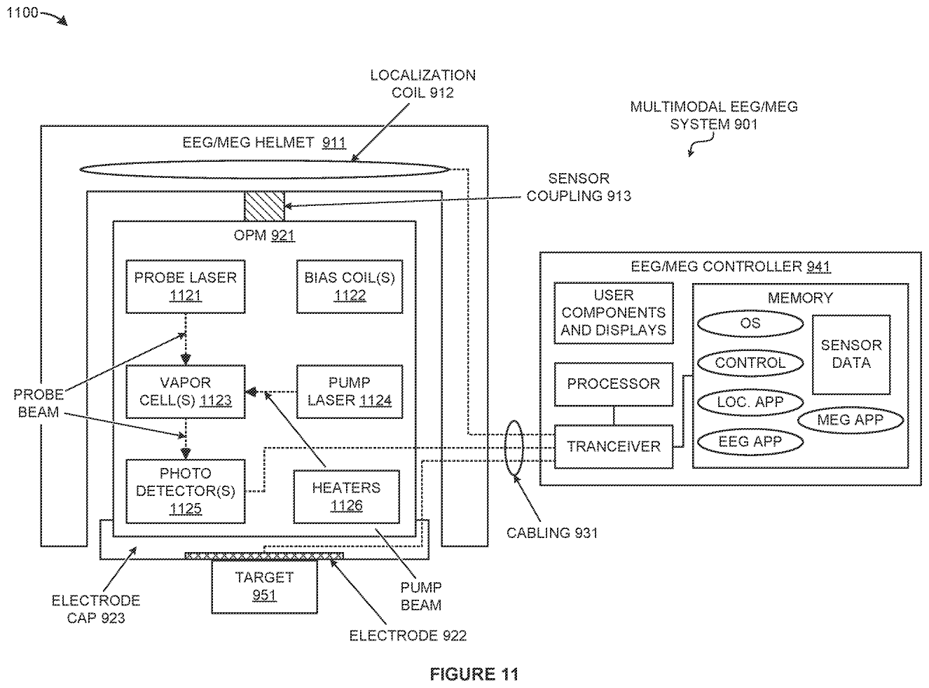

FIG. 11 comprises view 1100. View 1100 further illustrates multimodal EEG/MEG system 900. In this example, localization coils 912, sensor couplings 913, OPMs 921, electrodes 922, and electrode caps 923 are referred to in the singular for sake of clarity. OPM 921 comprises probe laser 1121, bias coil(s) 1122, vapor cell(s) 1123, pump laser 1124, photodetector(s) 1125, and heater(s) 1126. Target 951 is magnetically linked to OPM 921 and electrically linked to electrode 922. Localization coil 912 is magnetically linked to OPM 921 and metallically linked to cabling 931 which is metallically linked to controller 941. OPM 921 and electrode 922 are metallically linked to cabling 931 which is metallically linked to controller 941. Cabling 931 may be detachably coupled to controller 941. OPM 921 typically comprises other components like transceiver circuitry, signal processors, flash circuitry, thermal packaging, prisms, and/or other instruments and circuitry, however these additional components are omitted for clarity.

In some examples, vapor cell 1123 comprises an atomic device the encloses metallic vapor. Vapor cell 1123 may comprise a glass vapor cell, a silicon-glass vapor cell, and/or another type of vapor cell. The metallic vapor is selected for comprising properties relating to magnetic field sensitivity. Typically, the metallic vapor comprises an alkali metal vapor like rubidium vapor, cesium vapor, or potassium vapor. Vapor cell 1123 may enclose buffer gases (e.g., nitrogen or helium) in addition to the metallic vapor. Heaters 1126 comprise resistive heat elements operatively coupled to cell 1123. Heaters 1126 heat vapor cell 1123 to increase cell pressure. Bias coils 1122 comprise a set of coils arranged along the x, y, and z axes of vapor cell 1123. Bias coils 1122 emit a bias magnetic field to null or otherwise counteract background magnetic fields (e.g., the earths magnetic field) and/or to orient the sensing direction of OPM 921. Lasers 1121 and 1124 comprise lasers to optically pump and probe the metallic vapor housed by cell 1123. Probe laser 1121 and pump laser 1124 lasers may be combined in a single laser and/or additional lasers may be used. Photodetectors 1125 comprises a light sensor to absorb and measure the intensity of the beam emitted by probe laser 1121. When the probe beam passes through vapor cell 1123, a portion of the beam is absorbed by the metallic vapor. The remaining portion of the beam exists cell 1123 and is detected photodetector 1125. The amount of light absorbed by the metallic vapor correlates to the strength of the external magnetic field.

Controller 941 comprises transceiver circuitry (XCVR), memory, a processor, and user components and displays. The processor comprises a CPU, GPU, DSP, FPGA, ASIC, and/or some other type of processing circuitry. The memory comprises RAM, HDD, SSD, NVMe SSD, and the like. The memory stores software operating systems (OS), a control application, a localization (LOC.) application, a MEG application, and an EEG application. The processor retrieves the software from the memory and executes the software to drive the operation of the MEG system 901 as described herein. The operating system manages the hardware and software resources of controller 941. The control application controls the measurement operations of OPM 921 and electrode 922 and selects operating parameters (e.g., cell temperature) for OPM 921. The localization application determines the spatial location of OPM 921 and electrode 922 based on the measured field strength of the magnetic field generated by localization coil 912, the spatial location of coil 912 within helmet 911, and the orientational/positional constrains applied to OPM 921 its helmet slot and sensor coupling 913. The MEG application generates MEG images based on the measured magnetic field strength and the sensor location. The EEG application generates electroencephalograms based on the measured electric field strength and the sensor location. The sensor data comprises operating parameters of OPM 921 and electrode 922 like sensor Identifier (ID), slot ID, configuration parameters, and sensor performance metrics.

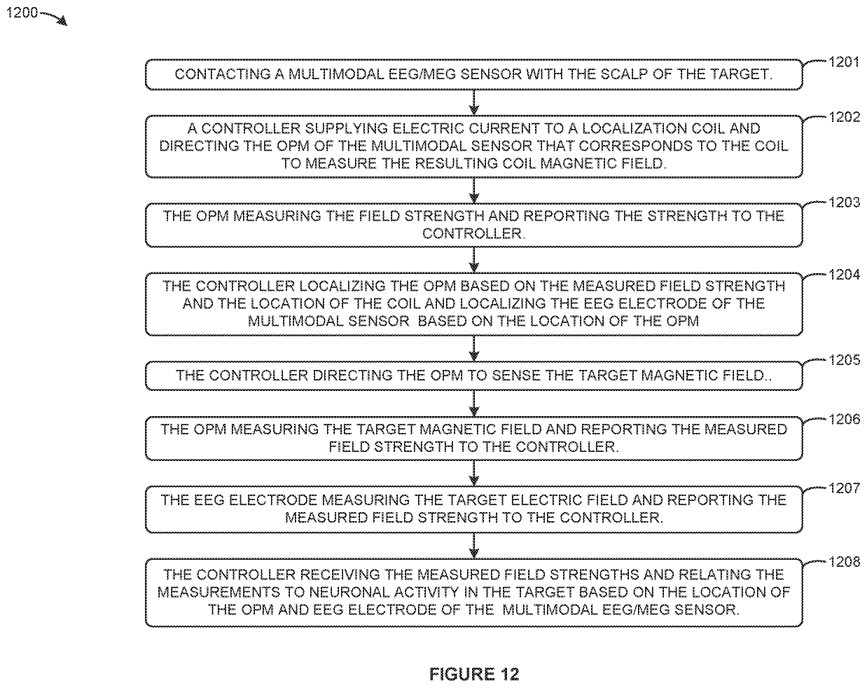

In some examples, EEG/MEG system 901 implements process 1200 illustrated in FIG. 12. It should be appreciated that the structure and operation of EEG/MEG system 901 may differ in other examples.

FIG. 12 illustrates process 1200. Process 1200 comprises an exemplary multimodal sensor localization and sensing process. In other examples, process 1200 may differ. The operations of process 1200 comprise contacting a multimodal EEG/MEG sensor with the scalp of the target (step 1201). The operations further comprise a controller supplying electric current to a localization coil and directing the OPM of the multimodal sensor that corresponds to the coil to measure the resulting coil magnetic field (step 1202). The operations further comprise the OPM measuring the field strength and reporting the strength to the controller (step 1203). The operations further comprise the controller localizing the OPM based on the measured field strength and the location of the coil and localizing the EEG electrode of the multimodal sensor based on the location of the OPM (step 1204). The operations further comprise the controller directing the OPM to sense the target magnetic field (step 1205). The operations further comprise the OPM measuring the target magnetic field and reporting the measured field strength to the controller (step 1206). The operations further comprise the EEG electrode measuring the target electric field and reporting the measured field strength to the controller (step 1207). The operations further comprise the controller receiving the measured field strengths and relating the measurements to neuronal activity in the target based on the location of the OPM and EEG electrode of the multimodal EEG/MEG sensor (step 1208).

Referring back to FIG. 11, EEG/MEG system 901 includes a brief example of process 1200 as employed by the various components of EEG/MEG system 901. In some examples, helmet 911 is placed on the head of target 951. An operator adjusts sensor coupling 913 (e.g., by twisting a set screw). Sensor coupling 913 moves OPM 921 and electrode 922 through their slot in helmet 911 until electrode 922 contacts the scalp of 951 (step 1201). The operator repeats the contacting process for the other OPMs/electrodes in helmet 911 until the sensor array is contoured to the scalp of target 911. Once contoured, the operator inputs a localization command to controller 941 via the user components and displays. The control application hosted by controller 941 receives the user input and selects OPM 921 and electrode 922 for localization. The control application correlates the sensor ID of OPM 921 to the coil ID of coil 912 and drives controller 941 to power coil 912.

Controller 941 supplies electrical current to coil 912 over cabling 931 (step 1202). Coil 912 generates magnetic waves that form a coil magnetic field. The magnitude of the magnetic field changes along the axis of motion of OPM 921. Typically, the measured strength of the coil magnetic field decreases as the distance between OPM 921 and coil 912 increases. Likewise, the measured strength of the coil magnetic field increases as the distance between OPM 921 and coil 912 decreases. Once coil 912 is powered, the control application transfers control signaling to OPM 921 that directs OPM 921 to measure the field strength of the coil magnetic field.

OPM 921 measures the field strength of the coil magnetic field in response to the control signals from controller 941 (step 1203). Vapor cell 1123 is positioned in the coil magnetic field. Bias coils 1122 generate bias fields to orient the sensing direction of vapor cell 1123. Heaters 1126 heat vapor cell 1123 to pressurize the metallic vapor. Pump laser 1124 emits a pump beam that is circularly polarized at a resonant frequency of the vapor to polarize the atoms. Probe laser 1121 emits a probe beam that is linearly polarized at a non-resonant frequency of the vapor to probe the atoms. The probe beam enters vapor cell 1123 where quantum interactions with the metallic vapor atoms in the presence of the coil magnetic field alter the energy/frequency of probe beam by amounts that correlate to the field strength of the coil magnetic field. Photodetectors 1125 detects the probe beam after these alterations by the metallic vapor atoms responsive to the coil magnetic field. Photodetector 1125 generates and transfers corresponding analog electronic signals that characterize the field strength of the coil magnetic field to controller 941 over cabling 931. In some examples, a signal processor (not shown) may filter, amplify, digitize, or perform other tasks on the analog electronic signals.

The control application in controller 941 receives the signals and directs the localization application to determine the spatial location of OPM 921 and electrode 922. The localization application correlates the field strength reported by OPM 121 to a distance between OPM 121 and coil 912. For example, the localization application may determine distance between OPM 921 and coil 912 is 24 millimeters based on the measured field strength. Although the example distance is given in millimeters, the localization application may operate on a more precise measurement scale like micrometers or nanometers. The localization application calculates the spatial location of OPM 921 and electrode 922 based on the correlated distance, the known location of coil 912 on helmet 911, and the orientational and positional constraints of OPM 921 and electrode 922 (step 1204). The localization application assigns the spatial location calculated for OPM 921 to electrode 922 as OPM 921 to electrode 922 are collocated by electrode cap 923. The localization application uses the spatial location of coil 912 and the orientation and location of the slot as reference points. For example, the localization application may execute a linearization function the receives the correlated distance, a direction vector of the slot, and a known spatial location of coil 912 as inputs and outputs the spatial location of sensor 921. The localization application stores the spatial location of OPM 921 and electrode 922 in memory as sensor data. The localization application notifies the control application that localization of OPM 921 and electrode 922 is complete. The control application drives controller 941 to stop powering coil 912 and selects a new OPM/electrode pair mounted to helmet 911 for localization.

Once all of OPM/electrode pairs on helmet 911 are located and their spatial locations are stored in the memory, the control application directs OPM 921 to measure the magnetic field generated by target 951 (step 1205). OPM 921 measures the field strength of the target magnetic field in response to the control signals from controller 941 (step 1206). Vapor cell 1123 is positioned in the target magnetic field. Bias coils 1122 generate bias magnetic fields to orient the sensing direction of vapor cell 1123. Heaters 1126 pressurize the metallic vapor and pump laser 1124 pumps the vapor to polarize the atoms. Probe laser 1121 probes the metallic vapor atoms. The probe beam interacts with the metallic vapor atoms in the presence of the target magnetic which alters the energy/frequency of probe beam by amounts that correlate to the field strength of the target magnetic field. Photodetectors 1125 detects the probe beam after these alterations and transfers analog electronic signals that characterize the field strength of the coil magnetic field to controller 941 over cabling 931.

Contemporaneously to the magnetic sensing operations of OPM 921, electrode 922 senses the electric field generated by target 951 (step 1207). The neuronal activity in the brain of target 951 results in the buildup of positive or negative electric charge via cellular ion exchange. When the intercellular space is negatively charged, the negative charge repels the electrons in the conductor of electrode 922. Likewise, when the intercellular space is positively charged, the positive charge attracts the electrons in the conductor of electrode 922. The pushing and pulling of the electrons are measured as voltage. Electrode 922 indicates this voltage to controller 941 over cabling 931. The other electrodes and other OPMs on helmet 911 also measure the electric and magnetic fields and report the measurements to controller 941 similarly to OPM 921 and electrode 922.

The control application in controller 941 receives the MEG data transferred by OPMs 921 and the EEG data transferred by electrodes 922. The control application directs the MEG application to generate an MEG image depicting the neuronal activity in target 951 and directs the EEG application to generate an electroencephalogram (step 1208). The MEG application processes the MEG data to generate a MEG image based on the detected field strengths of the target magnetic field and the spatial locations of OPMs 921. The EEG application process the EEG data to generate an electroencephalogram based on the measured field strengths of the target electric field and the spatial locations of electrodes 922. Controller 941 displays the resulting MEG image and electroencephalogram on the user interface for review by the operator.

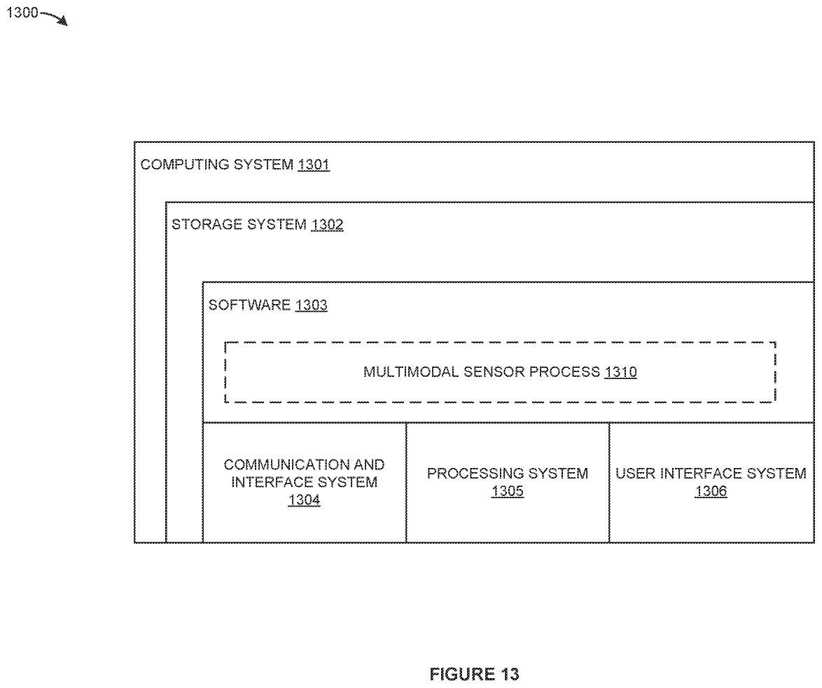

FIG. 13 illustrates computing environment 1300. Computing environment 1300 comprises computing system 1301. Computing system 1301 is representative of any system or collection of systems with which the various operational architectures, processes, scenarios, and sequences disclosed herein for performing multimodal sensor operations. For example, computing system 1301 may be representative of controller 331, controller 941, and/or any other computing device contemplated herein. Computing system 1301 may be implemented as a single apparatus, system, or device or may be implemented in a distributed manner as multiple apparatuses, systems, or devices. Computing system 1301 includes, but is not limited to, storage system 1302, software 1303, communication interface system 1304, processing system 1305, and user interface system 1306. Processing system 1305 is operatively coupled with storage system 1302, communication interface system 1304, and user interface system 1306.

Processing system 1305 loads and executes software 1303 from storage system 1302. Software 1303 includes and implements multimodal sensor process 1310, which is representative of any of the multimodal sensor processes described with respect to the preceding Figures, including but not limited to the sensor control, sensor localization, and sensor data processing operations described with respect to the preceding Figures. For example, multimodal sensor process 1310 may be representative of process 400 illustrated in FIG. 4, process 1000 illustrated in FIG. 10, and/or process 1200 illustrated in FIG. 12. When executed by processing system 1305 to implement a multimodal sensor operation, software 1303 directs processing system 1305 to operate as described herein for at least the various processes, operational scenarios, and sequences discussed in the foregoing implementations. Computing system 1301 may optionally include additional devices, features, or functionality not discussed for purposes of brevity.

Processing system 1305 may comprise a micro-processor and other circuitry that retrieves and executes software 1303 from storage system 1302. Processing system 1305 may be implemented within a single processing device but may also be distributed across multiple processing devices or sub-systems that cooperate in executing program instructions. Examples of processing system 1305 include general purpose CPUs, GPUs, DSPs, ASICs, FPGAs, analog computing devices, and logic devices, as well as any other type of processing device, combinations, or variations thereof.

Storage system 1302 may comprise any computer readable storage media readable by processing system 1305 and capable of storing software 1303. Storage system 1302 may include volatile, nonvolatile, removable, and/or non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1302 may also include computer readable communication media over which at least some of software 1303 may be communicated internally or externally. Storage system 1302 may be implemented as a single storage device but may also be implemented across multiple storage devices or sub-systems co-located or distributed relative to each other. Storage system 1302 may comprise additional elements, such as a controller, capable of communicating with processing system 1305 or possibly other systems.

Software 1303 (including multimodal sensor process 1310) may be implemented in program instructions and among other functions may, when executed by processing system 1305, direct processing system 1305 to operate as described with respect to the various operational scenarios, sequences, and processes illustrated herein. For example, software 1303 may include program instructions for localizing a multimodal EEG/MEG sensor, directing the multimodal EEG/MEG sensor to measure electric and magnetic fields generated by a target, and generating an MEG image and electroencephalogram depicting the neuronal activity of the target as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1303 may include additional processes, programs, or components, such as operating system software, virtualization software, or other application software. Software 1303 may also comprise firmware or some other form of machine-readable processing instructions executable by processing system 1305.

In general, software 1303 may, when loaded into processing system 1305 and executed, transform a suitable apparatus, system, or device (of which computing system 1301 is representative) overall from a general-purpose computing system into a special-purpose computing system customized to perform multimodal sensor operations as described herein. Indeed, encoding software 1303 on storage system 1302 may transform the physical structure of storage system 1302. The specific transformation of the physical structure may depend on various factors in different implementations of this description. Examples of such factors may include, but are not limited to, the technology used to implement the storage media of storage system 1302 and whether the computer-storage media are characterized as primary or secondary storage, as well as other factors.

For example, if the computer readable storage media are implemented as semiconductor-based memory, software 1303 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1304 may include communication connections and devices that allow for communication with other computing systems (not shown) over communication networks (not shown). Examples of connections and devices that together allow for inter-system communication may include network interface cards, antennas, power amplifiers, radiofrequency circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1301 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of networks, or variation thereof. The aforementioned communication networks and protocols are well known and an extended discussion of them is omitted for the sake of brevity.

While some examples provided herein are described in the context of computing devices for multimodal sensor processes, it should be understood that the multimodal sensors, multimodal sensing systems, and methods described herein are not limited to such embodiments and may apply to a variety of other environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

What is claimed is:

1. A multimodal electric and magnetic sensing apparatus to detect neuronal activity in a target, the apparatus comprising:
a magnetometer configured to sense a target magnetic field generated by the neuronal activity in the target; and
an electrode configured to contact a surface of the target and sense a target electric field generated by the neuronal activity in the target, wherein the electrode is coupled to the magnetometer.

2. The apparatus of claim 1 wherein the magnetometer comprises a Magnetoencephalography (MEG) Optically Pumped Magnetometer (OPM).

3. The apparatus of claim 1 wherein the electrode comprises an Electroencephalography (EEG) electrode.

4. The apparatus of claim 1 wherein the electrode is coupled to the magnetometer by at least one of an adhesive, a male/female socket connection, a threaded connection, or hook-and-loop fasteners.

5. The apparatus of claim 1 further comprising an electrode cap; and wherein:
the electrode is embedded into the surface of the electrode cap; and
the electrode cap is coupled to the magnetometer and is configured to contact the electrode to the surface of the target.

6. The apparatus of claim 5 wherein the electrode cap is coupled to the magnetometer by at least one of an adhesive, a male/female socket connection, a threaded connection, or hook-and-loop fasteners.

7. The apparatus of claim 1 wherein the electrode comprises a gold-plated electrode.

8. The apparatus of claim 1 wherein the electrode comprises a sponge electrode.

9. The apparatus of claim 1 wherein the electrode comprises a star shape geometry configured to inhibit eddy currents within the electrode.

10. A multimodal sensing system to detect neuronal activity in a target, the system comprising:
a sensor mount configured to mount a multimodal sensor array;
the multimodal sensor array comprising magnetometers and electrodes;
the magnetometers configured to sense a target magnetic field generated by the neuronal activity in the target;
the electrodes configured to contact a surface of the target and sense a target electric field generated by the neuronal activity in the target, wherein each of the electrodes is coupled to a corresponding one of the magnetometers;
a controller communicatively coupled to the magnetometers and the electrodes configured to process signaling received from the magnetometers and the electrodes to characterize the target magnetic field and the target electric field.

11. The multimodal sensing system of claim 10 wherein the magnetometers comprise Magnetoencephalography (MEG) Optically Pumped Magnetometers (OPMs) and the electrodes comprise Electroencephalography (EEG) electrodes.

12. The multimodal sensing system of claim 10 further comprising electrode caps configured to couple the electrodes to the magnetometers; and wherein:
the electrodes are embedded into the electrode caps; and
each of the electrode caps is coupled to the corresponding one of the magnetometers and are configured to contact the electrodes to the surface of the target.

13. The multimodal sensing system of claim 12 wherein the electrode caps are coupled to the magnetometers by at least one of an adhesive, a male/female socket connection, a threaded connection, or hook-and-loop fasteners.

14. The multimodal sensing system of claim 10 wherein the electrodes comprise gold plated electrodes.

15. The multimodal sensing system of claim 10 wherein the electrodes comprise sponge electrodes.

16. The multimodal sensing system of claim 10 wherein the electrodes comprise a surface geometry to inhibit eddy currents within the electrodes.

17. The multimodal sensing system of claim 16 wherein the surface geometry comprises a star shape.

18. The multimodal sensing system of claim 10 further comprising sensor localization coils; and wherein:
the sensor mount is configured to mount the multimodal sensor array and the sensor localization coils;
the controller is configured to supply electric current to the sensor localization coils;
the sensor localization coils are configured to receive the electric current and generate coil magnetic fields;
the magnetometers are configured to measure strengths of the coil magnetic fields;
the controller is configured to determine locations of the magnetometers based on the measured strengths of the coil magnetic fields and the spatial locations of the sensor localization coils; and
the controller is configured to determine locations of the electrodes based on the locations of the magnetometers.

19. A method of operating a multimodal sensing system to detect neuronal activity in a target, the method comprising:
a sensor mount mounting a multimodal sensor array comprising magnetometers and electrodes;
the magnetometers sensing a target magnetic field generated by the neuronal activity in the target;
the electrodes contacting a surface of the target and sensing a target electric field generated by the neuronal activity in the target, wherein each of the electrodes is coupled to a corresponding one of the magnetometers; and
a controller processing signaling received from the magnetometers and the electrodes to characterize the target magnetic field and the target electric field.

20. The method of claim 19 further comprising:
the sensor mount mounting sensor localization coils;
the controller supplying electric current to the sensor localization coils;
the sensor localization coils receiving the electric current and generating coil magnetic fields;
the magnetometers measuring strengths of the coil magnetic fields;
the controller determining locations of the magnetometers based on the measured strengths of the coil magnetic fields and the spatial locations of the sensor localization coils; and
the controller determining locations of the electrodes based on the locations of the magnetometers.
